(19) United States
(12) Patent Application Publication
(10) Pub. No.: US 2026/0266680 A1
(43) Pub. Date: Sep. 10, 2026

(54) Laser Fencing for Determining Fugitive Gas Emission

(71) Applicants: Saudi Arabian Oil Company, Dhahran (SA); King Abdullah University of Science and Technology, Thuwal (SA)

(72) Inventors: Adrian Cesar Cavazos Sepulveda, Thuwal (SA); Damian Pablo San Roman Alerigi, Al- Khobar (SA); Aamir Farooq, Thuwal (SA)

(21) Appl. No.: 19/070,006

(22) Filed: Mar. 4, 2025

(51) Int. Cl. G01M 3/04

(52) U.S. Cl. CPC G01M 3/04

(57) ABSTRACT

A method of determining gas emission from a facility. The method includes performing a series of optical measurements along one or more optical paths using a laser system, where the one or more optical paths defines an area of interest of the facility. The method also includes determining a total emission of a fugitive gas mixture from the area of interest based on the series of optical measurements using a flux form of Green's theorem. The laser system includes: a laser emitter to emit a laser; a reflector to reflect the laser; and a detector to receive and detect the laser reflected by the reflector.

### TECHNICAL FIELD

This document relates to laser fencing for determining fugitive gas emission.

### BACKGROUND

Fugitive gas emissions, unintended releases of gases from a facility, pose significant environmental and economic challenges. These emissions can include a variety of gases, including but not limited to methane (CH₄), volatile organic compounds (VOCs), and other hazardous air pollutants. The detection and quantification of these emissions are critical for environmental protection and regulatory compliance. Traditional methods of detection have relied on technologies such as infrared cameras and flame ionization detectors, which can be limited by their requirement for line-of-sight access and difficulties in quantifying low-level leaks. Advancements in technology are enhancing the ability to monitor and manage these emissions. Distributed sensing systems, for example, utilize a network of sensors and offer a comprehensive approach to detecting fugitive emissions across extensive industrial sites.

### SUMMARY

This disclosure describes technologies relating to systems and methods of laser fencing for determining fugitive gas emission from an oil and gas production facility. The laser fencing system can be based on an advanced photonic sensing system that includes laser emitters, reflectors, detectors, and a control system. The laser beam can pass through or surround the area of interest with potential gas emissions. This design allows for the approximate totalization of fugitive gas emissions using Green's theorem for 2D and the Divergence theorem for 3D. In 2D analysis, line integrals around a region's boundary convert to area integrals, facilitating total flux calculations. The laser fencing's versatile configurations enable applications in various facilities such as wellheads, pipelines, trunk lines, gas oil separation plants (GOSP), gas plants, and refineries.

An implementation described here provides a method of determining gas emission from a facility. The method includes performing a series of optical measurements along one or more optical paths using a laser system, where the one or more optical paths defines an area of interest of the facility. The method also includes determining a total emission of a fugitive gas mixture from the area of interest based on the series of optical measurements using a flux form of Green's theorem. The laser system includes: a laser emitter to emit a laser; a reflector to reflect the laser; and a detector to receive and detect the laser reflected by the reflector.

An implementation described here provides a laser fencing system for determining gas emission from a facility. The laser fencing system includes: a laser emitter to emit a laser; a reflector to reflect the laser; a detector to receive and detect the laser reflected by the reflector, where one or more optical paths of the laser defines an area of interest of the facility; and a computer processor including a non-transitory computer readable medium. The non-transitory computer readable medium stores instructions to: perform a series of optical measurements with the laser along the one or more optical paths; and determine an emission of a fugitive gas from the area of interest based on the series of optical measurements using a flux form of Green's theorem.

An implementation described here provides a laser fencing system for determining gas emission from a facility. The laser fencing system includes: a first laser emitter to emit a first laser; a plurality of reflectors to reflect the first laser; a detector to receive and detect the first laser; a second laser emitter to emit a second laser; a retroreflector to reflect the second laser back to a direction of the second laser emitter; a second detector positioned proximate to the second laser emitter and configured to receive and detect the second laser; and a computer processor including a non-transitory computer readable medium. The non-transitory computer readable medium stores instructions to: perform a series of optical measurements with each of the first and second lasers; and determine an emission of a fugitive gas from an area of interest based on the series of optical measurements using a flux form of Green's theorem.

### BRIEF DESCRIPTION OF THE DRAWINGS

For a more complete understanding of the implementations described herein, and the advantages thereof, reference is now made to the following descriptions taken in conjunction with the accompanying figures, in which:

FIG. 1 shows a schematic of a laser fencing system for fugitive gas emission monitoring, where the laser fencing horizontally surrounds an area of interest.

FIG. 2 shows a schematic of a laser fencing system for fugitive gas emission monitoring, where the laser fencing vertically surrounds an area of interest.

FIG. 3 shows a schematic of a laser fencing system for fugitive gas emission monitoring, where multiple parallel laser optical paths are used to form a laser curtain.

FIG. 4 shows a schematic of a laser fencing system for fugitive gas emission monitoring with sliders to move positions of a laser emitter and a reflector.

FIG. 5 shows a schematic of a laser fencing system for fugitive gas emission monitoring with a multicopter and a gimbal mounting a laser emitter for angular scanning.

FIG. 6 shows a schematic of a laser fencing system for fugitive gas emission monitoring that combines horizontal scanning and angular scanning.

FIG. 7 shows a schematic of a laser fencing system for fugitive gas emission monitoring that combines a horizontal laser fence and angular scanning.

FIG. 8 shows a schematic of a laser fencing system for fugitive gas emission monitoring that combines a horizontal laser fence and a pyramid configuration.

FIG. 9 shows a schematic of a circumferentially distributed reflector network for a laser fencing system.

FIG. 10 shows a diagram of a machine-learning model for fugitive gas emission monitoring.

FIG. 11 is a process flow diagram of an example method of determining fugitive gas emission using a laser fencing system.

### DETAILED DESCRIPTION

This disclosure describes systems and methods of laser fencing for determining fugitive gas emission from an oil and gas production facility. As an advanced photonic sensing system, the laser fencing system can include a set of one or more laser emitters, reflectors, and detectors, and a control system. The laser beam can be arranged to pass through or surround the area/space of interest at the facility with the possible gas emission, e.g., fugitive gas leakage. With the design of the laser fencing, it is possible to approximately totalize the quantity of the fugitive gas emissions by utilizing a combined form of the flux form of Green's theorem for two dimensions (2D) and the Divergence theorem for three dimensions (3D). In case of 2D analysis, one can convert line integrals around the boundary of a region to area integrals over the region, therefore facilitating calculations of total flux within a 2D area. The wide variety of configurations for the laser fencing can allow applications in various facilities/sites such as wellheads, pipelines, trunk lines, gas oil separation plant (GOSP), gas plants, and refineries.

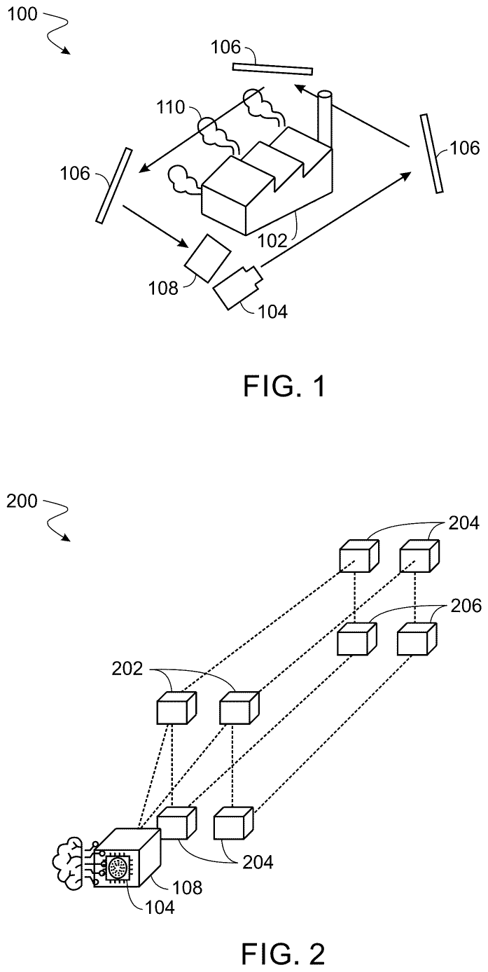

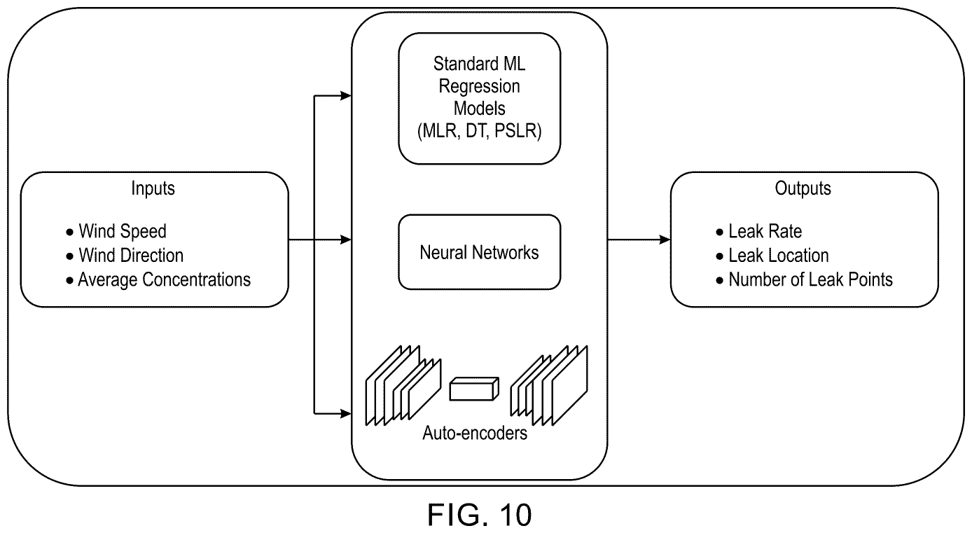

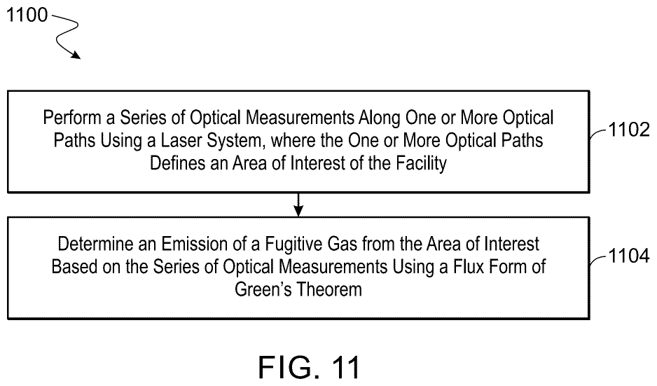

In the following, example designs and configurations for a laser fencing system are described referring to FIGS. 1-9. The application of a machine-learning model to further improve the accuracy of fugitive gas emission is described referring to FIG. 10. Methods of fugitive gas emission monitoring using a fencing system is then described referring to FIG. 11.

### Design of Laser Fencing System

FIG. 1 shows a schematic of a laser fencing system 100 for fugitive gas emission monitoring, where the laser fencing horizontally surrounds an area of interest of a facility 102. In various implementations, the laser fencing system 100 is suitable for detecting and quantifying fugitive gas emission from an oil and gas production facility. In some implementations, the facility 102 can be at any of upstream, midstream, or downstream applications. Accordingly, the facility 102 can include wellheads, pipelines, trunk lines, GOSP, gas plants, or refineries. In some implementations, the facility 102 can be a part of exploration, where gas seepage monitoring is of interest. Operational areas for the laser fencing system 100 can also be beyond oil and gas applications, extending to, for example, CO₂ geological storage and mining. Examples of the facility 102 include, but are not limited to GOSP, pipes, trunk lines, gas treatment units, sulfur removal units, refineries, factories, bio-factories and water desalination plants. Further examples could include the estimation of particulate pollution, and other contaminants, e.g. VOCs, produced within any given area.

While the laser fencing system 100 in FIG. 1 surrounds the facility 102 for illustration purposes, in various implementations, the fencing system 100 can be designed to monitor only a selected area within the facility 102. For example, the fencing system 100 can be designed to cover an area around certain sections of a pipeline within the facility 102, where the risk of gas leakage is predicted to be relatively higher than other sections of the facility 102.

In FIG. 1, in accordance with one implementation, the laser fencing system 100 includes a laser emitter 104, a set of reflectors 106, and a detector 108 for determining a fugitive gas 110 emitted from the facility 102. In various implementations, the laser emitter 104 can be Vertical-Cavity Surface-Emitting Laser (VCSEL), Interband Cascade Laser (ICL), Quantum Cascade Laser (QCL), or any other laser source. In some implementations, the laser with a single wavelength can be used. In some implementations, a combination or spectrum of wavelength can be used for the laser.

The laser from the laser emitter 104 can be used for optical measurements. In various implementations, the optical measurements are infrared absorption measurements, where the fugitive gas 110 has absorption at a wavelength used in the infrared absorption measurements. In some implementations, the absorption by the fugitive gas 110 is indicative of the total emission of the fugitive gas.

In various implementations, the fugitive gas 110 is a gas mixture. Further, the fugitive gas 110 can include at least one of C₁-C₅ hydrocarbons, carbon monoxide (CO), carbon dioxide (CO₂), benzene, toluene, xylene, or hydrogen disulfide (H₂S). In various implementations, the type of laser and the wavelength for the optical measurement are selected in view of the expected composition of the fugitive gas 110 so that the sensitivity of the optical measurement can be maximized.

In various implementations, the set of reflectors 106 are mirrors. In some implementations, as further described below referring to FIGS. 2-8, one or more of the reflectors can be retroreflectors. Retroreflectors are designed to reflect the incoming beam back to the direction of the beam source regardless of the incident angle.

As illustrated in FIG. 1, the laser emitter 104, the set of reflectors 106, and the detector 108 are positioned relative to each other so that the laser beam path forms a loop that defines the area of interest. In FIG. 1, the loop is a square that horizontally surrounds the facility 102. Configurations that form a loop allows the approximation based on special uses of Stokes general theorem in fugitive gas emission monitoring. Specifically, a combined form of the flux form of Green's theorem and the Divergence theorem can be applied for 2D and 3D approximation, respectively. Using these theorems, an order reduction in dimensionality is enabled, thereby allowing an approximation of complex engineering parameters such as fluid fluxes in a given area or volume. For example, when measuring the area of square, it is only required to know the length of all the sides, when measuring the volume of a cube, it is only required to know the faces of it.

In various implementations, the flux is approximated using ML, where the inputs are atmospheric parameters and concentrations. In some implementations, the measured concentration C can be converted to the flux F by approximating the flux from the time-derivative change of concentration. In some implementations, a basic weather model can be used to approximate the dispersion in a process akin to inversion.

In the following, the mathematical steps of flux determination are described in accordance with some implementations. In general, by conservation of mass, the total amount of the contaminant within the volume C_{Ω} is equal to the flux \vec{F} through the surface of that volume

$$\int \int \int {\partial }_{t} {C}_{Ω} dV = \int \int \vec{F} \cdot \vec{n} dA$$

This implies that the total amount of contaminant (Q) is related to the flux

$${\partial }_{t} Q = {\int }_{\partial Ω} \vec{F} \cdot \vec{n} dA$$

Since \vec{F} is directly related to the boundary concentrations (C_{σΩ}) via the method used to calculate the flux, its components are known. The total instantaneous flux and circulation can then be calculated. Accordingly, the method described in this disclosure can approximate the total amount of gas emission of a selected area or volume by only measuring the gas emission of their boundary, e.g., edges and faces, respectively.

The laser fencing system can be configured to provide a laser fence that horizontally encloses the area of interest as illustrated in FIG. 1. Accordingly, in FIG. 1, the laser emitter 104, the set of reflectors 106, and the detector 108 can be positioned at a same height from the ground or at a substantially same height, e.g., the height variance between the components is less than 20%, 15%, 10%, 5%, or 1%. In some implementations, the laser fence can vertically or slantly enclose the area of interest, as further described below referring to FIGS. 2, 3, and 5.

FIG. 2 shows a schematic of a laser fencing system 200 for fugitive gas emission monitoring, where the laser fencing vertically surrounds an area of interest. In FIG. 2, the laser fencing system 200 includes a laser emitter 104, a detector 108, a set of beam splitters 202, a set of mirrors 204, and a set of retroreflectors 206. In FIG. 2, using two sets of reflecting components, the laser fencing system 200 provides two vertical areas of interest, for illustration purposes, in accordance with one implementation. In various implementations, the number of area of interest a laser fencing system can provide is not limited to any number. Accordingly, in the following referring to FIG. 2, only one vertically enclosed area enabled by this configuration is described.

In FIG. 2, the beam splitter 202 splits the laser from the laser emitter 104 into a first laser and a second laser. The first laser travels horizontally to one of the mirrors 204 and is reflected downward to the retroreflector 206. The retroreflector 206 reflects the first laser back upward to the same one of the mirrors 204, and the first beam travels horizontally back to the beam splitter 202 and then to the detector 108. On the other hand, the second laser travels from the beam splitter 202 downward to a second one of the mirrors 204 and is reflected to travel horizontally to the retroreflector 206. The retroreflector 206 reflects the second laser back horizontally to the second one of the mirrors 204, and the second beam travels upward back to the beam splitter 202 and then to the detector 108.

The components (the laser emitter 104, the detector 108, the set of beam splitters 202, the set of mirrors 204, and the set of retroreflectors 206) are positioned so that two or more optical paths of the laser collectively form a loop the defines the area of interest. In FIG. 2, to vertically enclose the area of interest, the components are positioned at two different heights from the ground. In some implementations, although not specifically illustrated in FIG. 2, one or more components can be positioned at another height position. In FIG. 2, the detector 108 is at the substantially same position as the laser emitter 104, forming an integrated sensor system and enabling operating in reflection mode. In some implementations, the detector 108 can be positioned at a different position, for example, across the laser optical path to enable transmission mode.

In some implementations, selecting the area of interest vertically as illustrated in FIG. 2 can be beneficial where the fugitive gas can have a vertical gradient in concentration, e.g., near pipelines. For examples, dense gases such as H₂S (density≈1.36 kg/m³) tend to be in a higher concentration in a lower position in air (density≈1.29 kg/m³) contrary to other lighter gases such as CH₄ (density≈0.657 kg/m³) that tend to raise.

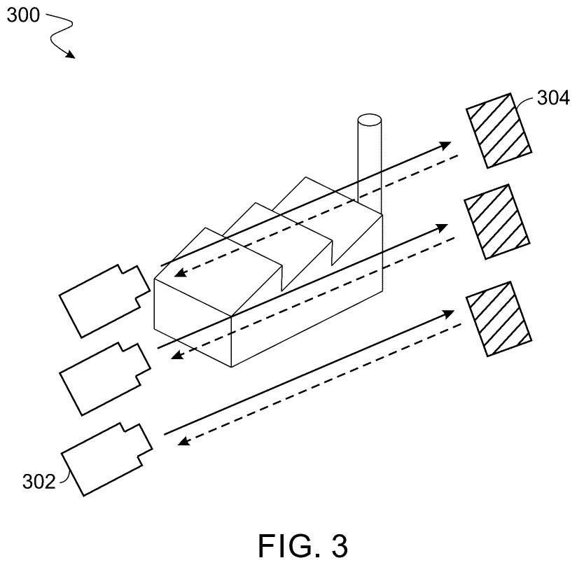

FIG. 3 shows a schematic of a laser fencing system 300 for fugitive gas emission monitoring, where multiple parallel laser optical paths are used to form a laser curtain.

In various implementations, a laser emitter and a detector can be coupled as an integrated device, for example as a laser sensor 302 as illustrated in FIG. 3. The integrated device can be used in the laser fencing system 300 in combination with a retroreflector 304. A single set of the laser sensor 302 and the retroreflector 304 can form a single optical path for the laser but may not be able to form a loop. Accordingly, in various implementations, multiple sets of the laser sensor 302 and the retroreflector 304 are used to from an array of optical paths. In some implementations, the laser fencing system 300 is configured to form an array of parallel optical paths as illustrated in FIG. 3. This array of optical paths can be termed a laser curtain and collectively define an area of interest for the fugitive gas emission monitoring.

While some implementations of fugitive gas emission determination may require an enclose surface or volume as illustrated in FIGS. 1-2, implementations of FIG. 3 may not provide such an enclosure defined by the laser optical paths. Accordingly, in this configuration, the area or volume of interest may be approximated by the model, in some case at the expense of some accuracy.

The array of optical paths can be used to determine the concentration gradient and estimate the gas flux using a time lapse and reducing the dependence or impact of the simplified atmospheric models. This could also be useful for physics informed and score-based machine learning (ML) models for totalization. Here, time lapse refers to the laser-based concentration measurements as a function of time. This time-dependent information can be coupled to environmental parameters. In various implementations, an ML model can be trained to calculate leak rates/leak locations using such time-dependent information.

In various implementations, the laser curtain can be arranged vertically as illustrated in FIG. 3, horizontally, or slantly. Having an angle of the laser curtain can help measuring emission existing the facility 102 in the upward direction. In some implementations, the laser fencing system 300 can be configured to provide more than one laser curtains. In some implementations, the concept of laser curtain can be enabled by incorporating a moving mechanism to enable spatial scanning of the laser.

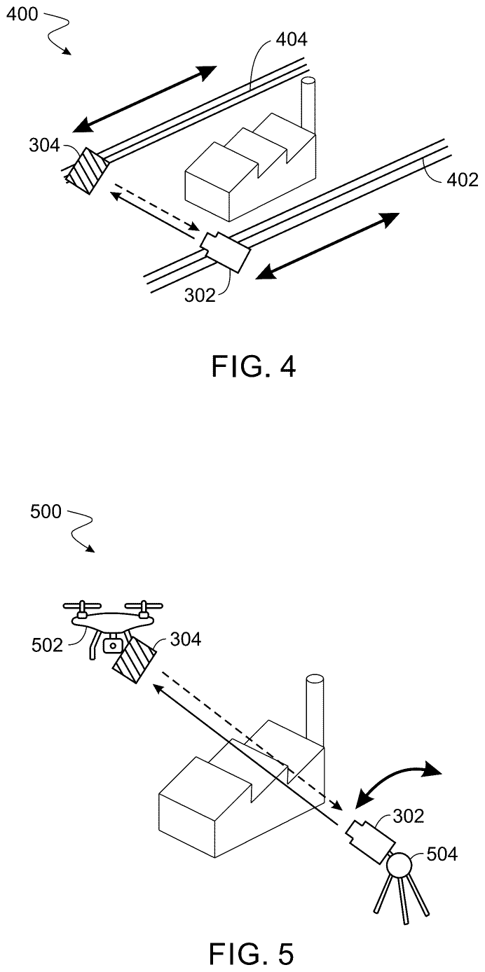

FIG. 4 shows a schematic of a laser fencing system 400 for fugitive gas emission monitoring with sliders to move positions of a laser sensor 302 and a reflector 304. In FIG. 4, the laser fencing system 400 includes a laser slider 402 that mounts the laser sensor 302 to slide the laser sensor 302, and a reflector slider 404 that mounts the retroreflector 404 to slide the retroreflector 404. Accordingly, the configuration of the laser fencing system 400 enables spatial scanning of the laser to form a temporal laser curtain by performing a series of optical measurements at different locations along the sliders as indicated by arrows in FIG. 4. In various implementations, the temporal laser curtain can be arranged horizontally as illustrated in FIG. 4, vertically, or slantly.

FIG. 5 shows a schematic of a laser fencing system 500 for fugitive gas emission monitoring with a multicopter 502 carrying a retroreflector 304 and a gimbal 504 mounting a laser sensor 302 for angular scanning. The gimbal 504 can allow the laser sensor 302 to be faced to track and point to the multicopter 502. This configuration can be used primarily for slant spatial scanning of the laser to form the temporal laser curtain.

In some implementations, the combination of the laser sensor 302 and the gimbal 504 can be enhanced or replaced with an integrated system such as a fish-eye or Fresnel type of optical arrangement. The gimbal tracking can be based on feedback from a focal plane array (FPA) that images the returning laser from the retroreflector 304.

Further, in one or more implementations, a high-power light emitting diode co-aligned with the laser can also be used to enhance the tracking of the retroreflector 304. The FPA images can be processed to find the retroreflector location via 2D peak finding. As a result, the gimbal 504 can be directed to track and follow the location of the retroreflector 304.

Although not specifically illustrated in FIG. 5, instead of using a multicopter, the retroreflector 304 can be fixed at a position at a relatively high position on a high post or already available structure of the facility 102, for example, a roof. With separating a laser emitter and a detector, this configuration can form a pyramid-like optical arrangement with an area of interest in a triangular shape slantly positioned.

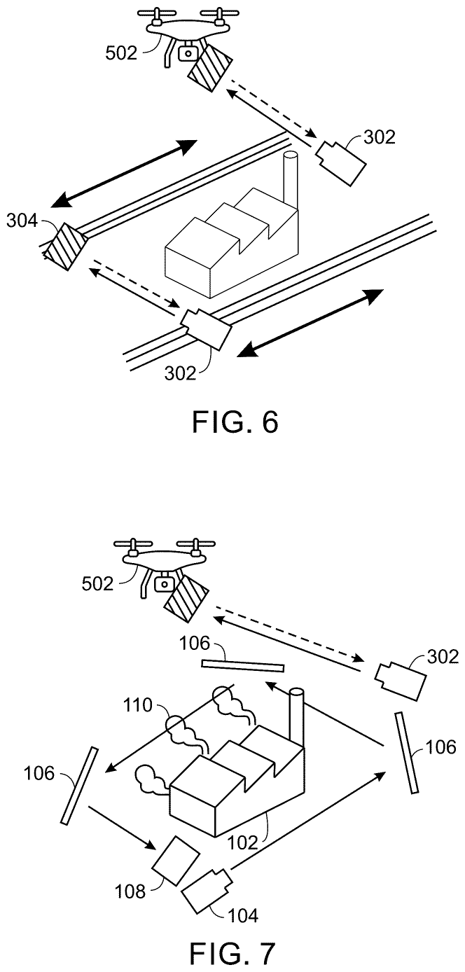

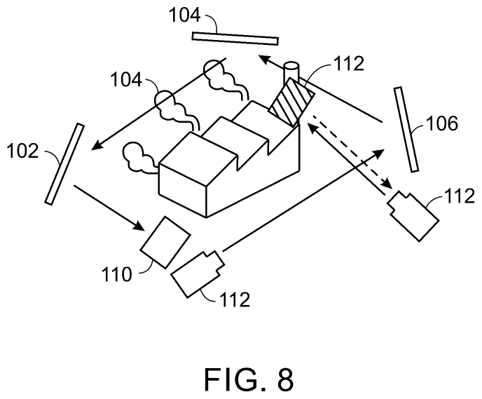

In some implementations, the above-mentioned laser fencing approaches can be used in combination to further enhance the accuracy and flexibility of the laser fencing system. horizontal scanning and angular scanning. In some implementations, the horizontal scanning (e.g., FIG. 4) and the angular scanning (e.g., FIG. 5) are combined (FIG. 6). In some implementations, the horizontal laser fence (e.g., FIG. 1) and the angular scanning (e.g., FIG. 5) are combined (FIG. 7). In some implementations, the horizontal laser fence (e.g., FIG. 1) and the pyramid configuration can be combined (FIG. 8).

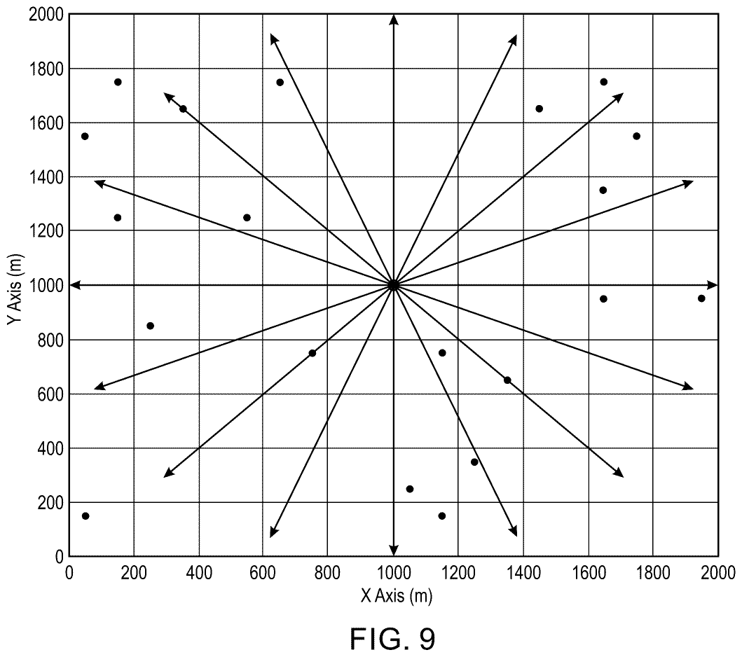

FIG. 9 shows a schematic of a circumferentially distributed reflector network for of a laser fencing system. In some implementations, the laser sensor can be configured to be swept angularly to direct the laser to a network of retroreflectors circumferentially distributed. This can be configured to cover a majority or an entire region of possible areas of gas leak. In FIG. 9, there are sixteen lines that indicate the laser beam directions originating from the center where the laser course is located. Sixteen triangles indicate the fixed retroreflectors distributed within the facility. Circles indicate leak locations.

In some implementations, the radial sweeping can be performed using a 360° rotating base, e.g., a gimbal, to mount the laser emitter and the detector, or an integrated laser sensor. The retroreflectors are fixed at predetermined locations with predetermined angles and circumferentially distributed. In some implementations, a fish-eye or Fresnel type of arrangement is used.

Implementations of the combined use of multiple fencing approaches as described referring to FIG. 6-8 are for examples, and other configurations are also possible. Any fencing approaches described referring to FIGS. 1-5 and 9 can be combined with any number of other approaches to enable a multi-fencing configuration.

### Use of Machine-Learning Model

FIG. 10 shows a diagram of a machine-learning model for fugitive gas emission monitoring. In various implementations, the algorithm used for determining the fugitive gas emission is enhanced using a machine-learning (ML) model. Examples of standard ML regression models include Multiple Linear Regression (MLR), Decision Tree (DT), and Partial Least Squares Regression (PLSR). Other types of ML models such as Synthetic Data Vault (SDV) and neural network models. Examples of the neural network models include Convolutional Neural Network (CNN), Variational Autoencoder (VAE), Long Short-Term Memory (LSTM), and Transformers. The input data to the ML model can include the optical measurement data for gas emissions and environmental parameters such as wind speed, wind direction, humidity, atmospheric pressure, solar radiation, and precipitation. The ML model can generate outputs such as leak rate, leak location, and the number of leak points. As a preliminary step, the ML model can be trained using a set of training data set. In some implementations, the training data set includes historical data of environmental parameters, historical data of emissions of the fugitive gas from the area of interest, or both. Further, in some implementations, geospatial data and 3D models are additionally used to improve the leak location. Geospatial data refers to information that describes objects, events, or phenomena with a location on the surface of the Earth, and it can indicate locations of the potential leak sources, e.g., gas wells, pipelines, or gas plants.

The method of determining fugitive gas emission can be performed with or without a ML model. In various implementations, regardless of the use of a ML model, the method can use relevant models and data such as plume emission models and atmospheric dispersion modeling for different gases or mixtures of gases to approximate the gas flux.

### Method of Fugitive Gas Emission Monitoring

FIG. 11 is a process flow diagram of an example method of determining fugitive gas emission using a laser fencing system. A process 1100 starts with a step 1102 of performing a series of optical measurements along one or more optical paths using a laser system. An area of interest of the facility is defined by the one or more optical paths. The laser system can include a laser emitter to emit a laser, a reflector to reflect the laser, and a detector to receive and detect the laser reflected by the reflector. Subsequently, at a step 1104, a total emission of a fugitive gas mixture from the area of interest is determined based on the series of optical measurements using a flux form of Green's theorem.

In some implementations, the series of optical measurements is a first series of optical measurements performed at a first wavelength, and the method further includes performing a second series of optical measurements at a second wavelength. The total emission of a fugitive gas mixture can then be determined based on both of the first and second series of optical measurements.

To determine leak locations and leak rates, in various implementations, the method can further include factoring in environmental parameters such as such as wind speed, wind direction, humidity, atmospheric pressure, solar radiation, and precipitation. Further, the averaged concentrations in the path-length of each laser beam can also be considered. Absorbance signals along different radial directions and the corresponding averaged concentrations can be calculated using the Beer-Lambert law. Subsequently, the relationship between the averaged measured concentrations and the leakage information can be established using an atmospheric transport model that describes how the leakage affects the concentration distribution. Then, leakage locations and rates can be determined based on such a relationship.

In various implementations, the fugitive gas emission monitoring not only detect a single species but also several species by using a detection algorithm enhanced with machine learning and scanning of the laser frequency (wavelength). Further, the laser fencing system of this disclosure can be used to monitor real time operational data of a facility remotely and periodically. While other commercial technologies such as a point of measurement technology or line measurement technologies suffer from procedural measurement biases or the wind direction, the laser fencing system of this disclosure can circumvent some of these challenges by approximating a 2D type of measurement to a fencing type of measurement.

### Implementations

An implementation described here provides a method of determining gas emission from a facility. The method includes performing a series of optical measurements along one or more optical paths using a laser system, where the one or more optical paths defines an area of interest of the facility. The method also includes determining a total emission of a fugitive gas mixture from the area of interest based on the series of optical measurements using a flux form of Green's theorem. The laser system includes: a laser emitter to emit a laser; a reflector to reflect the laser; and a detector to receive and detect the laser reflected by the reflector.

In an aspect, the series of optical measurements is based on infrared absorption measurements, the fugitive gas mixture has absorption at a wavelength used in the infrared absorption measurements, and the absorption by the fugitive gas mixture is indicative of the total emission of the fugitive gas mixture.

In an aspect, combinable with any other aspect, the series of optical measurements is performed using a single wavelength.

In an aspect, combinable with any other aspect, the series of optical measurements is a first series of optical measurements performed at a first wavelength, the method further including performing a second series of optical measurements at a second wavelength, where the total emission of a fugitive gas mixture is determined based on both of the first and second series of optical measurements.

In an aspect, the first series of optical measurements provides at least information about an emission of a first component of the fugitive gas mixture, and the second series of optical measurements provides at least information about an emission of a second component of fugitive gas mixture.

In an aspect, combinable with any other aspect, the method further includes scanning a wavelength of the laser during the series of optical measurements.

In an aspect, combinable with any other aspect, the fugitive gas mixture includes methane.

In an aspect, combinable with any other aspect, the fugitive gas mixture includes at least one of C₁-C₅ hydrocarbons, carbon monoxide (CO), carbon dioxide (CO₂), benzene, toluene, xylene, or hydrogen disulfide (H₂S).

In an aspect, combinable with any other aspect, a single optical path of the series of optical measurements forms a loop that defines the area of interest.

In an aspect, combinable with any other aspect, two or more optical paths of the series of optical measurements collectively form a loop the defines the area of interest.

In an aspect, combinable with any other aspect, the series of optical measurements is performed along an array of parallel optical paths.

In an aspect, combinable with any other aspect, the laser emitter and the detector are positioned proximate to each other, and the reflector includes a retroreflector to reflect back the laser to a direction of the laser emitter.

In an aspect, combinable with any other aspect, the series of optical measurements laser includes spatially scanning the laser emitter.

In an aspect, combinable with any other aspect, the series of optical measurements laser includes spatially scanning the reflector.

In an aspect, combinable with any other aspect, the series of optical measurements laser includes changing a steering angle of the laser emitter for angular scanning.

In an aspect, combinable with any other aspect, the series of optical measurements laser includes changing a location of the reflector.

In an aspect, the laser system further includes a multicopter carrying the reflector.

In an aspect, combinable with any other aspect, the method further includes: monitoring an environmental condition proximate to the area of interest; and updating the emission of the fugitive gas based on the environmental condition.

In an aspect, the environmental condition includes wind speed, wind direction, humidity, atmospheric pressure, solar radiation, or precipitation.

In an aspect, the method further includes: training a machine-learning model using a set of training data set including historical data of environmental conditions and historical data of emissions of the fugitive gas from the area of interest; and using the trained machine-learning model to updating the emission of the fugitive gas.

An implementation described here provides a laser fencing system for determining gas emission from a facility. The laser fencing system includes: a laser emitter to emit a laser; a reflector to reflect the laser; a detector to receive and detect the laser reflected by the reflector, where one or more optical paths of the laser defines an area of interest of the facility; and a computer processor including a non-transitory computer readable medium. The non-transitory computer readable medium stores instructions to: perform a series of optical measurements with the laser along the one or more optical paths; and determine an emission of a fugitive gas from the area of interest based on the series of optical measurements using a flux form of Green's theorem.

In an aspect, the reflector is a first mirror, the laser fencing system further including a second mirror to reflect the laser, where an optical path of the laser defined by the laser emitter, the first and second mirrors, and the detector forms a loop that defines the area of interest.

In an aspect, combinable with any other aspect, the reflector is a retroreflector, the laser fencing system further including: a beam splitter to receive and split the laser from the laser emitter, forming a first laser and a second laser; a first mirror positioned to reflect the first laser to the retroreflector; and a second mirror positioned to reflect the second laser to the retroreflector. The retroreflector is configured to reflect the first laser back to the first mirror and reflect the second laser back to the second mirror, and optical paths of the first laser and the second laser collectively form a loop that defines the area of interest.

In an aspect, combinable with any other aspect, the reflector is a first reflector, the laser emitter is a first laser emitter, the laser is a first laser, and the detector is a first detector, the laser fencing system further including: a second laser emitter to emit a second laser; a second reflector to reflect back the second laser to a direction of the second laser emitter, and a second detector proximate to the second laser emitter and configured to receive and detect the second laser reflected by the second reflector. An optical path of the first laser and an optical path of the second laser are parallel, and the non-transitory computer readable medium stores further instructions to perform another series of optical measurements with the second laser along one or more optical paths.

In an aspect, combinable with any other aspect, the reflector is a retroreflector, and the laser fencing system further includes: a laser slider mounting the laser emitter to slide the laser emitter; and a reflector slider mounting the retroreflector to slide the retroreflector.

In an aspect, combinable with any other aspect, the reflector is a retroreflector, and the laser fencing system further includes a gimbal mounting the laser emitter to angularly scan the laser.

In an aspect, the laser fencing system further includes a multicopter mounting the retroreflector.

In an aspect, combinable with any other aspect, the reflector is positioned on a roof of the facility.

In an aspect, combinable with any other aspect, the laser fencing system further includes a sensor to monitor an environmental condition proximate to the area of interest, where the non-transitory computer readable medium stores further instructions to update the emission of the fugitive gas based on the environmental condition.

An implementation described here provides a laser fencing system for determining gas emission from a facility. The laser fencing system includes: a first laser emitter to emit a first laser; a plurality of reflectors to reflect the first laser; a detector to receive and detect the first laser; a second laser emitter to emit a second laser; a retroreflector to reflect the second laser back to a direction of the second laser emitter; a second detector positioned proximate to the second laser emitter and configured to receive and detect the second laser; and a computer processor including a non-transitory computer readable medium. The non-transitory computer readable medium stores instructions to: perform a series of optical measurements with each of the first and second lasers; and determine an emission of a fugitive gas from an area of interest based on the series of optical measurements using a flux form of Green's theorem.

While the disclosure has been shown in some of its forms, it should be apparent to those skilled in the art that it is not so limited, but is susceptible to various changes and modifications without departing from the scope of the disclosure based on experimental data or other optimizations considering the overall economics of the process. Accordingly, it is appropriate that the appended claims be construed broadly and in a manner consistent with the scope of the disclosure.

What is claimed is:

1. A method of determining gas emission from a facility, the method comprising:
performing a series of optical measurements along one or more optical paths using a laser system, the one or more optical paths defining an area of interest of the facility; and
determining a total emission of a fugitive gas mixture from the area of interest based on the series of optical measurements using a flux form of Green's theorem,
wherein the laser system comprises:
a laser emitter to emit a laser;
a reflector to reflect the laser; and
a detector to receive and detect the laser reflected by the reflector.

2. The method of claim 1, wherein the series of optical measurements is based on infrared absorption measurements, the fugitive gas mixture has absorption at a wavelength used in the infrared absorption measurements, the absorption by the fugitive gas mixture is indicative of the total emission of the fugitive gas mixture.

3. The method of claim 1, wherein the series of optical measurements is performed using a single wavelength.

4. The method of claim 1, wherein the series of optical measurements is a first series of optical measurements performed at a first wavelength, the method further comprising performing a second series of optical measurements at a second wavelength, wherein the total emission of a fugitive gas mixture is determined based on both of the first and second series of optical measurements.

5. The method of claim 4, wherein the first series of optical measurements provides at least information about an emission of a first component of the fugitive gas mixture, and the second series of optical measurements provides at least information about an emission of a second component of fugitive gas mixture.

6. The method of claim 1, further comprising scanning a wavelength of the laser during the series of optical measurements.

7. The method of claim 1, wherein the fugitive gas mixture comprises methane.

8. The method of claim 1, wherein the fugitive gas mixture comprises at least one of C₁-C₅ hydrocarbons, carbon monoxide (CO), carbon dioxide (CO₂), benzene, toluene, xylene, or hydrogen disulfide (H₂S).

9. The method of claim 1, wherein a single optical path of the series of optical measurements forms a loop that defines the area of interest.

10. The method of claim 1, wherein two or more optical paths of the series of optical measurements collectively form a loop the defines the area of interest.

11. The method of claim 1, wherein the series of optical measurements is performed along an array of parallel optical paths.

12. The method of claim 1, wherein the laser emitter and the detector are positioned proximate to each other, and the reflector comprises a retroreflector to reflect back the laser to a direction of the laser emitter.

13. The method of claim 1, wherein the series of optical measurements laser comprises spatially scanning the laser emitter.

14. The method of claim 1, wherein the series of optical measurements laser comprises spatially scanning the reflector.

15. The method of claim 1, wherein the series of optical measurements laser comprises changing a steering angle of the laser emitter for angular scanning.

16. The method of claim 1, wherein the series of optical measurements laser comprises changing a location of the reflector.

17. The method of claim 16, wherein the laser system further comprises a multicopter carrying the reflector.

18. The method of claim 1, further comprising:
monitoring an environmental condition proximate to the area of interest; and
updating the emission of the fugitive gas based on the environmental condition.

19. The method of claim 18, wherein the environmental condition comprises wind speed, wind direction, humidity, atmospheric pressure, solar radiation, or precipitation.

20. The method of claim 18, further comprising:
training a machine-learning model using a set of training data set comprising historical data of environmental conditions and historical data of emissions of the fugitive gas from the area of interest; and
using the trained machine-learning model to updating the emission of the fugitive gas.

21. A laser fencing system for determining gas emission from a facility, the laser fencing system comprising:
a laser emitter to emit a laser;
a reflector to reflect the laser;
a detector to receive and detect the laser reflected by the reflector, wherein one or more optical paths of the laser defines an area of interest of the facility; and
a computer processor comprising a non-transitory computer readable medium storing instructions to:
perform a series of optical measurements with the laser along the one or more optical paths; and
determine an emission of a fugitive gas from the area of interest based on the series of optical measurements using a flux form of Green's theorem.

22. The laser fencing system of claim 21, wherein the reflector is a first mirror, the laser fencing system further comprising a second mirror to reflect the laser, wherein an optical path of the laser defined by the laser emitter, the first and second mirrors, and the detector forms a loop that defines the area of interest.

23. The laser fencing system of claim 21, wherein the reflector is a retroreflector, the laser fencing system further comprising:
a beam splitter to receive and split the laser from the laser emitter, forming a first laser and a second laser;
a first mirror positioned to reflect the first laser to the retroreflector; and
a second mirror positioned to reflect the second laser to the retroreflector, wherein the retroreflector is configured to reflect the first laser back to the first mirror and reflect the second laser back to the second mirror, and
optical paths of the first laser and the second laser collectively form a loop that defines the area of interest.

24. The laser fencing system of claim 21, wherein the reflector is a first reflector, the laser emitter is a first laser emitter, the laser is a first laser, and the detector is a first detector, the laser fencing system further comprising:
a second laser emitter to emit a second laser;
a second reflector to reflect back the second laser to a direction of the second laser emitter; and
a second detector proximate to the second laser emitter and configured to receive and detect the second laser reflected by the second reflector,
wherein an optical path of the first laser and an optical path of the second laser are parallel, and
the non-transitory computer readable medium stores further instructions to perform another series of optical measurements with the second laser along one or more optical paths.

25. The laser fencing system of claim 21, wherein the reflector is a retroreflector, the laser fencing system further comprising:
a laser slider mounting the laser emitter to slide the laser emitter; and
a reflector slider mounting the retroreflector to slide the retroreflector.

26. The laser fencing system of claim 21, wherein the reflector is a retroreflector, the laser fencing system further comprising a gimbal mounting the laser emitter to angularly scan the laser.

27. The laser fencing system of claim 26, further comprising a multicopter mounting the retroreflector.

28. The laser fencing system of claim 21, wherein the reflector is positioned on a roof of the facility.

29. The laser fencing system of claim 21, further comprising a sensor to monitor an environmental condition proximate to the area of interest, wherein the non-transitory computer readable medium stores further instructions to update the emission of the fugitive gas based on the environmental condition.

30. A laser fencing system for determining gas emission from a facility, the laser fencing system comprising:
a first laser emitter to emit a first laser;
a plurality of reflectors to reflect the first laser;
a detector to receive and detect the first laser;
a second laser emitter to emit a second laser;
a retroreflector to reflect the second laser back to a direction of the second laser emitter;
a second detector positioned proximate to the second laser emitter and configured to receive and detect the second laser; and
a computer processor comprising a non-transitory computer readable medium storing instructions to:
perform a series of optical measurements with each of the first and second lasers; and
determine an emission of a fugitive gas from an area of interest based on the series of optical measurements using a flux form of Green's theorem.
